(19) United States
(12) Patent Application Publication
(10) Pub. No.: US 2012/0115201 A1
(43) Pub. Date: May 10, 2012

(54) Methods and Systems for Producing Biomass and/or Biotic Methane Using an Industrial Waste Stream

(76) Inventor: D. Jack Adams, Park City, UT (US)

(21) Appl. No.: 13/256,206

(22) PCT Filed: Mar. 12, 2010
(86) PCT No.: PCT/US10/27156

§ 371 Date: Nov. 22, 2011

Related U.S. Application Data
(60) Provisional application No. 61/160,261, filed on Mar. 13, 2009.
(60) Provisional application No. 61/237,656, filed on Aug. 27, 2009.
(60) Provisional application No. 61/245,427, filed on Sep. 24, 2009.

(51) Int. Cl. C12P 5/02; C12N 1/20; C12M 1/00; C12N 1/12

(52) U.S. Cl. 435/167; 435/257.1; 435/252.1; 435/289.1

(57) ABSTRACT

Systems and methods for capture and sequestration of CO₂, SOₓ, NOₓ, and/or other compounds present in industrial waste streams and use of the captured waste as a nutrient source to support a microbial population. The microbial population can use the nutrients from the industrial waste stream and an energy source such as sunlight or a hydrocarbon deposit for the production of biomass, heavy metals recovery, and the generation of methane and other biogases. Biomass can be processed to produce a variety of hydrocarbon fuels, lubricants, and the like.

### CROSS-REFERENCE TO RELATED APPLICATIONS

This application claims the benefit of and priority to U.S. Provisional Patent Application Ser. No. 61/160,261 filed 13 Mar. 2009, U.S. Provisional Patent Application Ser. No. 61/237,656 filed 27 Aug. 2009, and U.S. Provisional Patent Application Ser. No. 61/245,427 filed 24 Sep. 2009, the entireties of which are incorporated herein by reference.

### BACKGROUND

The need exists to reduce and/or eliminate CO₂, SOₓ, NOₓ and other emissions from industrial operations such as power plants. The need is particularly acute in the coal industry and other industries that use carbon-based fossil fuel sources. One alternative to coal is coal bed methane (CBM). The existence of methane in coal beds has been recognized for many years. The first commercial production of coal bed methane probably occurred in the eastern United States in the 1920's or 1930's. Recently, coal bed methane production has increased dramatically and now represents approximately 10% of U.S. domestic gas production.

Production of coal bed methane typically involves drilling a well down into a deep layer of coal and injecting a fluid into the bed at high pressure to fracture the coal. Fracturing the coal along with removal of the water in the seam allows trapped methane to escape from the coal bed. The coal in these deep coal reserves is plentiful, but often not accessible.

Worldwide, coal resources that are not mineable may constitute more than 7 trillion tons, which is larger than all the oil and gas, oil shale and oil sands combined. These resources present a potential untapped energy source. An important feature of developing this resource is carbon management.

The long-term use of power derived from traditional fossil fuel resources is questionable due to production of net carbon dioxide, SOₓ, NOₓ, and other pollutants that are linked to global climate change, acid rain, and other environmental maladies. Other industrial waste streams are also under heavy scrutiny from environmental agencies throughout the world. The search for suitable methods to treat industrial waste streams continues to be a long-felt but unmet need.

### SUMMARY

The technology disclosed herein relates to the capture and sequestration of CO₂, SOₓ, NOₓ, and/or other compounds present in the waste streams from industrial processing plants such as, but not limited to, coal fired power plants, petroleum refineries, and food processing plants. The methods disclosed herein relate to processes for growing a microbial population using the industrial waste stream as a source of nutrients for the microbes. In one aspect, the microbial population can, for example, include microbes capable of producing methane using nutrients from the industrial waste stream and an energy source such as sunlight or a hydrocarbon deposit. In another aspect, the microbial population can be configured to produce a biomass that can be processed to produce a hydrocarbon fuel.

In one embodiment of the present invention, a system for bio-energy recovery and/or production is disclosed. The system includes water source, an industrial waste stream including nutrient compounds capable of producing a nutrient rich water when admixed with water, and a population of microorganisms including algae, archaea, bacteria, methanogenic microorganisms, pH tolerant microbes, heat tolerant microbes, and/or brine tolerant microbes, and combinations thereof. The population of microorganisms is configured to propagate on the nutrient rich body of water and produce a bio-energy product (e.g., a biomass) therefrom. The industrial waste stream is capable of providing a chemical environment (e.g., nutrient components, particulates, etc. in the nutrient rich water) that can be adapted for modifying and selecting the relative density of various microbes in the microbial population, increasing microbial yields for biomass production, and/or increasing biotic methane production.

Suitable examples of water sources include, but are not limited to, natural and artificial lakes, ponds, engineered water systems optimized for microbial and algal production, and the like and generally moist environments that can support microbial growth and/or algal growth. In one aspect, the water source can be a surface body of water such as a pond, a lake, or a waste lagoon. In another aspect, the water source can be a subterranean body of water such as a body of water associated with a geological formation that includes a hydrocarbon material. Waters associated with geological formations that include hydrocarbon materials include waters that are naturally associated with the geological formation (e.g., an aquifer) or waters introduced into the geological formation.

In another embodiment of the present invention, a method includes (1) providing an industrial waste stream including nutrient compounds capable providing a nutrient rich environment when mixed with water, (2) mixing the industrial waste stream with a water source to produce a nutrient rich water, (3) propagating a population of microorganisms including at least one of algae, archaea, bacteria, methanogenic microorganisms, pH tolerant microbes, heat tolerant microbes, and/or brine tolerant microbes in the nutrient rich water, and (4) recovering a bio-energy product produced by the population of microorganisms.

In yet another embodiment of the present invention, a method includes (1) providing a body of water, (2) providing an industrial waste stream including nutrient compounds capable of providing a nutrient rich environment when mixed with water, (3) mixing the industrial waste stream with the body of water to produce a nutrient rich body of water, (4) propagating a bacterial and/or algal population in the nutrient rich body of water in the presence of sunlight, wherein the bacterial and/or algal population includes algae, archaea, bacteria, methanogenic microorganisms, pH tolerant microbes, heat tolerant microbes, and/or brine tolerant microbes, and combinations thereof, and (5) recovering a biological product from the nutrient rich body of water for use as an energy source.

In still yet another embodiment of the present invention, a method for producing biotic methane is disclosed. The method includes (1) providing an industrial waste stream including nutrient compounds capable of providing a nutrient rich environment when mixed with water, (2) providing a geological formation that includes a hydrocarbon material, (3) providing a water source within the geological formation, (4) mixing the industrial waste stream with the water source to produce a nutrient rich water, (5) providing a microbial population and/or one or more microbial components within the geological formation, (6) allowing the microbial population to propagate and produce biotic methane using the hydrocarbon material and the nutrient rich water, and (7) recovering at least a portion of the biotic methane from the geological formation. Methane production in a geological formation typically involves the breakdown of complex organic molecules by different populations of microbes into simpler molecules that can be utilized by methanogens for production of methane. Microbes, cellular components (e.g., surfactants and enzymes), and nutrients can be added to the geological formation in a staged and/or cyclic manner in order to maximize methane production in the geological formation.

In one aspect, the microbial population provided within geological formation includes at least one of archaea, bacteria, methanogenic microorganisms, pH tolerant microbes, heat tolerant microbes, and/or brine tolerant microbes, and combinations thereof. According to the present invention, the microbial population provided within the geological formation can be a microbial population that is indigenous to the geological formation or indigenous to a similar geological formulation, or the microbial population can be augmented microbial consortia that is propagated in the nutrient rich body of water and subsequently injected in whole or in part into the geological formation. The microbial population can, for example, be injected into the geological formation in a liquid water or as an aerosol.

In another aspect, the one or more microbial components include enzymes and surfactants adapted to facilitate breakdown of complex organics into the simple carbon compounds needed for biotic methane production and/or microbial growth in the geological formation. The one or more microbial components (e.g., enzymes and surfactants) are typically derived from the microbial population.

In yet another one aspect, the water source provided within the geological formation can be a surface water that is injected into the geological formation, or the body of water can be a subsurface body of water that is associated with the geological formation.

These and other objects and features of the present invention will become more fully apparent from the following description and appended claims, or may be learned by the practice of the invention as set forth hereinafter.

### BRIEF DESCRIPTION OF THE DRAWINGS

To further clarify the above and other advantages and features of the present invention, a more particular description of the invention will be rendered by reference to specific embodiments thereof which are illustrated in the appended drawings. It is appreciated that these drawings depict only illustrated embodiments of the invention and are therefore not to be considered limiting of its scope. The invention will be described and explained with additional specificity and detail through the use of the accompanying drawings in which:

FIG. 1 illustrates a flow diagram of a system for producing a biomass and/or biotic methane using an industrial waste stream and a microbial population;

FIG. 2 illustrates a flow diagram of a system for bio-energy recovery and/or production using an industrial waste stream and a microbial population;

FIG. 3A illustrates a schematic diagram of a system for producing biotic methane in a geological formation;

FIG. 3B is a schematic diagram illustrating the biotic conversion of carbon polymers in coal, oil shale, tar sands, heavy oils, and the like into simpler compounds that are then converted to methane;

FIG. 4 illustrates a flow diagram of a method for bio-energy recovery and/or production using an industrial waste stream and a microbial population;

FIG. 5 illustrates a flow diagram of a method for bio-energy recovery and/or production using an industrial waste stream, sunlight, and a microbial population; and

FIG. 6 illustrates a flow diagram of a method for producing biotic methane in a geological formation using an industrial waste stream and microbial populations.

### DESCRIPTION

The technology disclosed herein relates to the capture and sequestration of CO₂, SOₓ, NOₓ, and/or other compounds present in the waste streams from industrial processing plants such as, but not limited to, coal fired power plants, petroleum refineries, mineral processing, and food processing plants. The methods disclosed herein relate to processes for growing a microbial population using the industrial waste stream as a source of nutrients for the microbes. In one aspect, the microbial population can, for example, include microbes capable of producing methane using nutrients from the industrial waste stream and an energy source such as sunlight or a hydrocarbon deposit. In another aspect, the microbial population can be configured to produce a biomass that can be processed to produce a hydrocarbon fuel.

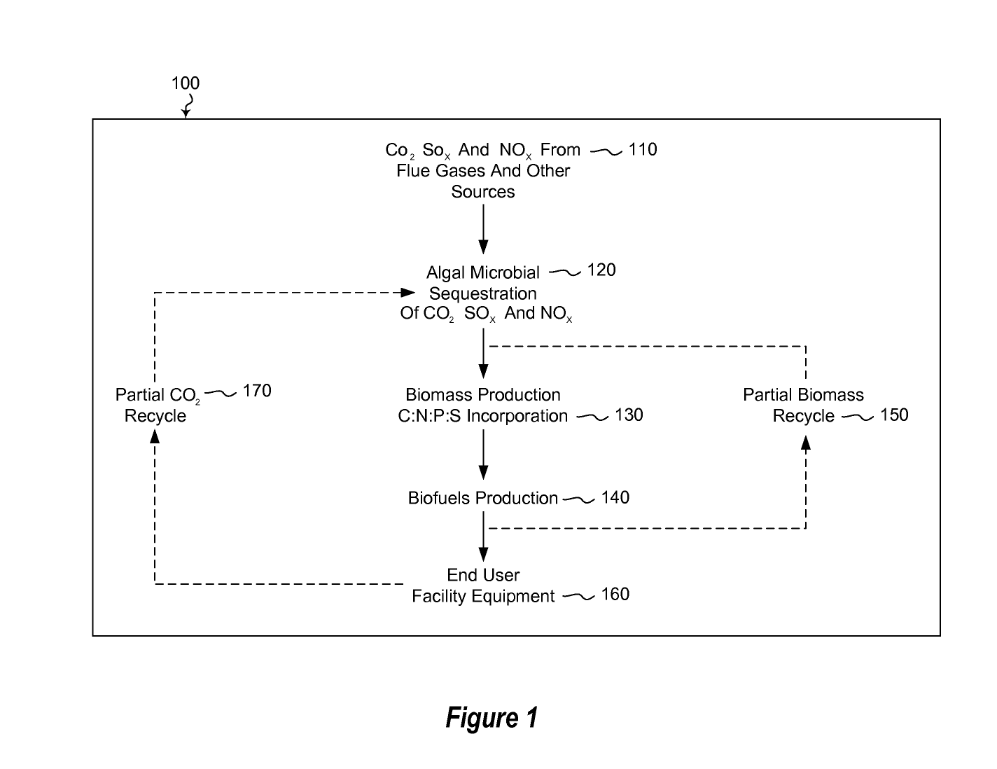

FIG. 1 generally describes a system 100 for sequestering CO₂, SOₓ, and NOₓ from a flue gas or other source using a microbial population. As shown in FIG. 1, the system 100 includes a flue gas 110. Suitable sources of flue gases include, but are not limited to coal fired power plants, factories, and other industrial operations. The flue gas 110 includes CO₂, SOₓ, and NOₓ, metals and other compounds typically found in the flue gas from fossil fuel combustion. Flue gases are typically discharged into the atmosphere as waste. However, flue gases are rich in minerals and compounds that can be used as a nutrient source for supporting microbial growth. For example, the flue gas 110 can be injected into a body of water such as a pond or engineered system using gas/water mixing devices whereby the CO₂, SOₓ, and NOₓ are dissolved and/or suspended in the water. A portion of the components of the flue gas 110 dissolve into the body of water and increase the salt concentration of the water. If the dissolved salts are sufficiently concentrated, the dissolved salts form a brine water.

As further shown in FIG. 1, the system 100 includes an algal and/or bacteria population 120 that can utilize carbon, nitrogen phosphorus, and sulfur derived from the flue gas 110 to grow and thereby form a biomass 130 in the nutrient rich body of water. As shown in FIG. 1, the biomass 130 is recovered and processed to form a biofuel 140. In another embodiment (not shown), the algal and/or bacteria population 120 can be configured or adapted to produce methane gas that can be used directly as a biofuel.

According the illustrated embodiment of the system 100, biofuels production 140 produces a waste stream that is then recycled back into the body of water as indicated by 150. The biofuel is delivered to an end user 160 that utilizes the fuel (e.g., by burning the fuel), thereby producing carbon dioxide. The carbon dioxide can be captured and directly recycled or the carbon dioxide can be released into the air and indirectly recycled through absorption by the body of water.

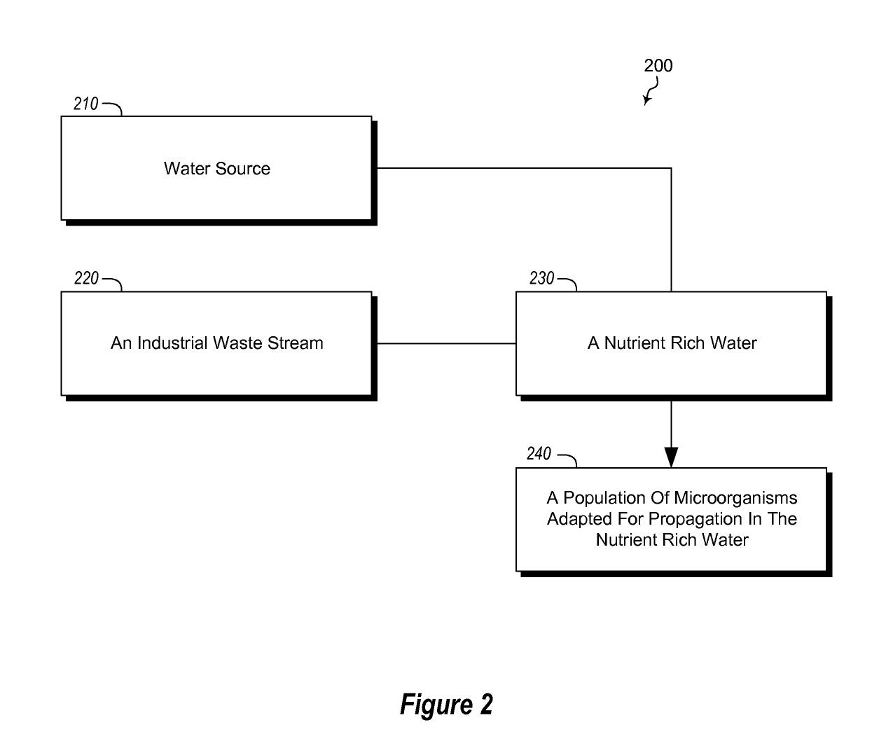

Referring now to FIG. 2 system 200 for bio-energy recovery and/or production is schematically illustrated. The system 200 includes a water source 210 and an industrial waste stream 220. The industrial waste stream 220 includes nutrient compounds capable of producing a nutrient rich water 230 when the industrial waste stream 220 is admixed with the water source 210. The illustrated system further includes a population of microorganisms 240 that are configured to propagate in the nutrient rich water 230. According to one embodiment, the population of microorganisms 240 includes at least one of algae, archaea, bacteria, methanogenic microorganisms, pH tolerant microbes, heat tolerant microbes, and/or brine tolerant microbes that are tolerant to high salt and/or high ionic strength.

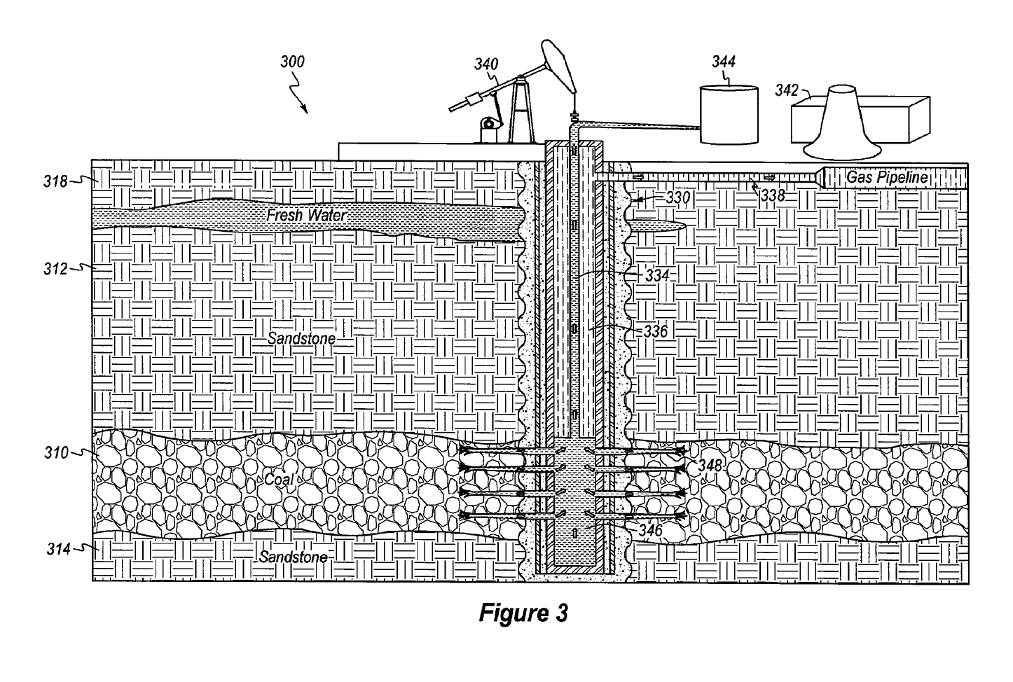

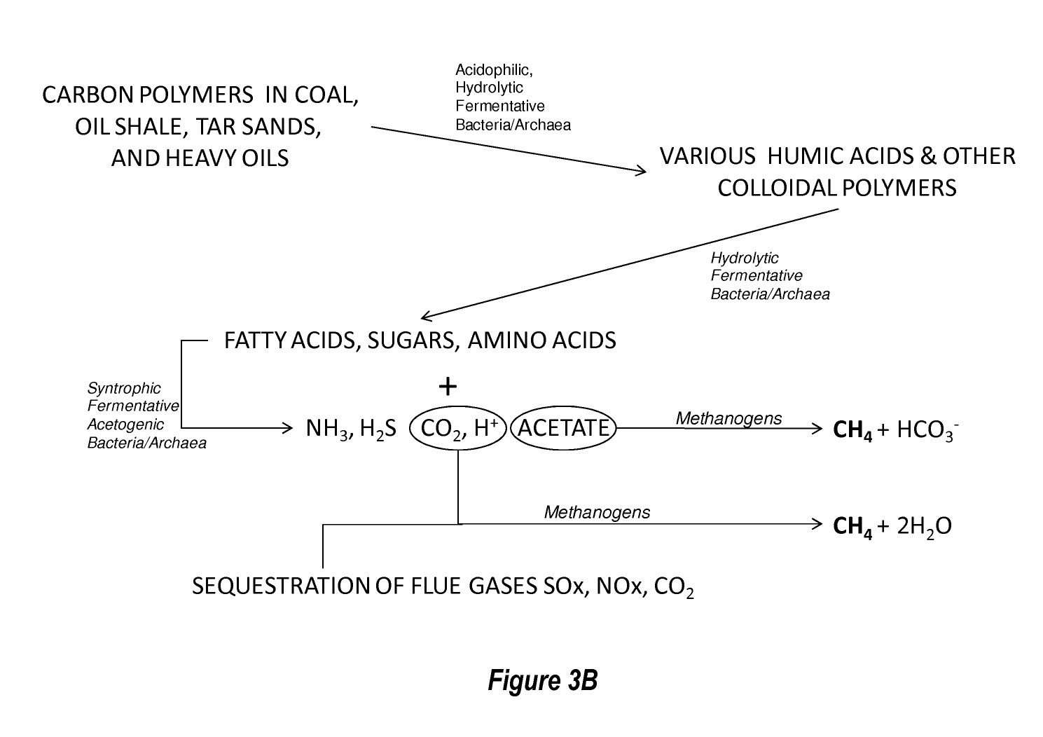

Suitable examples of water sources 210 include, but are not limited to, natural and artificial lakes, ponds, and the like and generally moist environments that can support microbial growth. In one aspect, the water source 210 can be a surface body of water such as a pond, a lake, or a waste lagoon. In another aspect, the water source 210 can be a subterranean body of water such as a body of water associated with a geological formation (see, e.g., FIG. 3A) that includes a hydrocarbon material.

According to one embodiment of the system, the industrial waste stream 220 is a gaseous waste stream such as a flue gas. Suitable examples of flue gases include, but are not limited to, a flue gas from an industrial power plant, a steel mill, a factory, and the like. In one aspect, the flue gas is flue gas from a coal fired power plant. According to another embodiment, the industrial waste stream is a liquid waste stream such as from a food processing plant.

Referring now to FIG. 3A, schematic diagram 300 of a system for producing biotic methane in a geological formation is illustrated. Natural gas generated from microbial activity in natural organic deposits of coal, oil shale, oil sands, depleted oil fields, and heavy oils represent an increasingly important natural resource. It is estimated that natural gas from microbial activity (methanogenesis) currently accounts for about 20% of the world's natural gas resource and represents a potentially greatly expandable resource. In general, methane from these sources comes from both thermogenic and biogenic sources. High temperatures generate thermogenic methane from deeply buried coal. Biogenic methane is formed from microbial degradation of complex organics to form simple molecules, such as hydrogen and carbon dioxide and molecules like acetate that, in combination with other nutrient components, fuel microbial growth, metabolism and the generation of methane.

Coal is extremely rich in complex organic matter. Under the right conditions, many constituents in coal, oil shales, oil sands, and heavy oils contain carbon and other nutrient components and are biodegradable. Coal is, however, solid rock containing a mixture of many complex lignin- and organic-derived compounds having an overall chemical formula that is approximated by C₁₃₅H₉₆O₉NS that can be resistant to degradation. The rate-limiting step of coal biodegradation is typically the initial fragmentation and degradation of the macromolecular lignin network of coal and complex by products formed. Lignin degradation can be achieved by fungi and some microbes and a significant quantity of coals can be broken-down using extracted microbial enzymes (e.g., broken microbial cells).

Biochemical processes of complex carbon degradation with microorganism participation include several types of enzyme reactions based on oxygenases, dehydrogenases, and hydrolases, and some acid production. The enzyme reactions cause aromatic and aliphatic hydrooxidation, oxidative deamination, hydrolysis, and other biochemical transformations of the original complex carbon substances and the intermediate products of their degradation. Surfactants can also be important in the separation of the products produced and in producing an environment in which degradation of complex hydrocarbons can occur. The degree and rates of hydrocarbon biodegradation depend, first of all, upon the structure of their molecules. With increasing complexity of molecular structure (i.e., increasing the number of carbon atoms and degree of chain branching) as well as with increasing molecular weight, the rate of microbial decomposition usually decreases.

Most of the coal bed methane (CBM) existing today was formed millions of years ago, in an environment that does not exist in the bulk of CBM deposits today. Therefore, different microbial populations and environmental conditions are required for degradation of the “new” carbon sources now available. Methane is an end product of a complex series of interactions in which several groups of non-pathogenic bacteria work together to utilize organic carbon, producing methane.

Many constituents in coal, oil shales, oil sands, and heavy oils contain carbon and other nutrient components and are biodegradable. The main limitations to their actual biodegradation are combinations of water, temperature, pH, salinity, surface area, oxidation reduction potential (ORP), adequate and appropriate microbial populations, and nutrient component bioavailability. All microbes require nutrient components including carbon, nitrates, phosphates, sulfates, and others in proper ratios in order to grow and metabolize the organics in complex hydrocarbon fuel sources. An imbalance in these ratios slows and eventually halts microbial growth and metabolism. Natural geological formations that have coal, oil shales, oil sands, and/or heavy oils are sufficiently imbalanced so as to preserve the hydrocarbon fuel source.

With specific reference now to FIG. 3A, a system is illustrated for producing biotic methane in a fossil fuel-bearing geological formation 310 using a water source, an industrial waste stream, and a population of microorganisms. Geological formation 310 is sandwiched between sedimentary rock layers 312 and 314. Geological formation 310 can be at any depth, but in one embodiment is at a depth that is impractical for extracting using traditional mining techniques. In one embodiment, the geological formation 310 can be a newly discovered and/or newly developed geological formation or, alternatively, the geological formation can be the source for an abandoned well or a low producing well in which increased methane production is desired. The geological formation 310 may also be an old coal mine, depleted oil-field, or other developed resource that includes residual hydrocarbon resources that were not extracted (usually due to a lack of feasibility).

Geological formation 310 can be a dry formation or include an aquifer. For example, the geological formation may include a saline aquifer. In one embodiment, the saline water can include calcium, magnesium, sulfates, sodium chloride, and/or metal salts. In another embodiment, water 348 (e.g., water from a terrestrial source) can be injected into the geological formation 310 by well component 346. The water 348 can act to crack the geological formation 310 and/or act a support medium for microbial growth in the geological formation 310. Water containing nutrients/biochemicals and/or microbes can be injected into the geological formation 310 as liquid water, water vapor, or as an aerosol.

Above layer 314 is a fresh water aquifer 318. Residential well 320 may be used to supply residential home 322 with fresh water. An extraction well 330 is formed through aquifer 318 and sandstone layer 312 in order to obtain access to geological formation 310. Well 330 has a cement casing 332 that protects fresh water aquifer 318 from water and gases in geological formation 310.

Geological formation 310 can be prepared for enhanced methane production by fracturing the hydrocarbon to increase its surface area. In one embodiment, a fluid, such as water is pumped into geological formation 310 using a pump 340 to fracture the coal in geological formation 310. The fluid in conduit 334 may be any fluid such as but not limited to water and/or mixtures of water and nutrient producing fluids such as flue gas from coal fired power plant 342. The fracturing fluid may be stored, blended, and otherwise prepared using pumps and blenders known in the art. In one embodiment, the fracturing fluid and/or nutrient containing fluid is stored in tank 344 prior to injection into geological formation 310.

In one embodiment, geological formation 310 may include an indigenous microbial population and/or nutrients needed to break down the hydrocarbon in the geological formation to form methane. For example, the indigenous population of microorganisms may include at least one methanogenic microorganism capable of producing a biotic methane in the subterranean geological formation using the industrial waste stream and the hydrocarbon material. In another example, the indigenous population of microorganisms may include at least one microorganism that is adapted to produce a methane precursor material for the at least one methanogenic microorganism.

While the entire microbial population and/or all the necessary nutrients may be injected using extraction well 330, in many cases, a portion of the microbial population and/or the nutrients will already exist in the geological formation. Thus, the system typically includes injecting the proper types and amounts of microbial populations and rate limiting nutrients that will increase the production of methane as compared to the naturally occurring microbial activity. The microbial population, nutrients, and/or fracturing fluid can be injected together or separately in any number of injection steps. The microbial population and/or nutrients and/or water can be injected into the geological formation as a liquid water (e.g., in a slurry) or as an aerosol. In one aspect, aerosols can be advantageous due to their ability disperse widely in the geological formation 310

In one embodiment, the microbes injected into the geological formation are extremeophiles. The microorganisms may be provided by culturing naturally occurring bacteria and archaea under the conditions in which biotic methane will be produced. The adjusted conditions can facilitate self selection of microbial populations that are most suited for producing complex carbon compound breakdown and subsequent production of methane in the harsh environment of the geological formation. Different combinations of bacteria and archaea populations can be used in the selection process. Microorganisms are the earliest forms of life on earth and occupy almost every conceivable ecological niche, even the harshest, most extreme, and toxic environments. About 1 billion microbes live in a single teaspoon of moist garden soil. In a preferred embodiment, the microbial population is enriched with extremophiles including halophiles and thermophiles, which are microbes that thrive in high salt and high-temperature environments, respectively. Using a variety of naturally occurring bacteria and archaea in combination with selection pressures allows the most robust population of microbes to be obtained. However, the systems and methods disclosed herein may also be carried out using engineered microbes alone or in combination with naturally occurring microbes. Engineered microbes include microbes produced through classical selection methods or other genetic modification methods. Where an engineered microbe is used, the engineered microbes may be produced from one or more of the foregoing naturally occurring microbes that have been selected to provide robust methane production in the geological formation.

Microbes require certain nutrients to grow and multiply. For example, microbes require a carbon source, nitrates, phosphates, sulfates, potassium, magnesium, iron, and various other elements and compounds. The rate of growth of a microbial culture can and often is limited by the concentration of one or more nutrients essential for growth. Some of the most important nutrients for growing microbes are present in the waste streams of industrial processes. For example, carbon, nitrates, phosphates, sulfates, and trace metals are present in the flue gas of coal fired power plants. Currently, industrial plants expend a significant amount of resources to separate and extract the foregoing compounds from the waste streams. In one embodiment, the systems methods described herein avoid many of the costs incurred to dispose of industrial waste streams by using the waste stream as a nutrient rich stream for growing microbes in a geological formation.

The following describes various components of the system in greater detail. All or a portion of the following features described below can be used to sequester CO₂, SOₓ, and NOₓ, manufacture a biomass, and/or produce a biotic methane.

### A. An Industrial Waste Stream

The biomass and/or biotic methane are produced using an industrial waste. Industrial waste streams (e.g., flue gas from a coal fired power plant) are generally discharged into the environment as waste or are recovered inefficiently by air scrubbers and the like. However, industrial waste streams contain valuable nutrients such as carbon, nitrates, sulfates, and the like that can be used to support microbial growth.

Microbes require certain nutrients to grow and multiply. For example, microbes require a carbon source, nitrates, phosphates, sulfates, potassium, magnesium, iron, and various other elements and compounds. The rate of growth of a microbial culture can and often is limited by the concentration of one or more nutrients essential for growth. Some of the most important nutrients and environmental adjustment biochemicals for growing algae, bacteria, and archaea are present in the waste streams of industrial processes. For example, carbon, nitrates, and sulfates, are present in the flue gas of coal fired power plants. Currently, industrial plants expend a significant amount of resources to separate and extract the foregoing compounds from the waste streams. In contrast, the methods described herein avoid many of the costs incurred to dispose of industrial waste streams by using the waste stream as a nutrient rich stream for growing algae and bacteria. That is, using materials in the waste stream to support microbial growth efficiently recovers the resources in the waste stream and can efficiently sequester CO₂, SOₓ, NOₓ, heavy metals, and other pollutants that may be harmful if discharged.

The industrial waste streams used in the systems and methods described herein can be used in combination with other sources of nutrients to provide a desired growing environment for the microbes. For example, the industrial waste stream can be mixed with or injected into a body of water and used to support microbial growth and or to adjust the environment to make it more suitable for microbial growth. In some embodiments, the body of water into which the nutrient stream is injected together with the industrial waste stream will be a non-ideal nutrient concentration. Thus, in one embodiment, supplemental nutrients and/or a combination of two or more industrial waste streams can be used to produce the desired nutrient concentrations. Additional nutrients can be added as a concentrated liquid or powder to the industrial waste stream and/or to the body of water in which the microbes will be growing.

The nutrients can be added to the system to optimize the nutrient concentrations for obtaining high growth rates of the algae, bacteria, and/or archaea. In one embodiment, the nutrient rich stream is added to a body of water to provide a carbon concentration of about 100-120 mg/l, a nitrogen concentration of about 10-20 mg/l, a phosphorus concentration of about 1-3 mg/l, a sulfur concentration of about 1-2 mg/l and/or a combination of any of the foregoing. Providing the nutrients within the foregoing ranges and/or ratios may ensure rapid growth rates.

In many embodiments, the industrial waste stream is provided at a temperature above ambient temperature. Most industrial processes are operated at elevated temperatures and the waste stream from these processes includes a significant amount of heat that cannot be economically recovered in the industrial process. For example, flue gas from a coal fired power plant is usually relatively warm. In one embodiment, the industrial waste stream has a temperature that is warmer than ambient and/or is warmer than the body of water into which the industrial waste stream is injected. Thus, the industrial waste stream provides a source of heating for maintaining a desired temperature for growing the algae and/or bacteria. In a preferred embodiment, the industrial waste stream has a temperature greater than about 20° C., more preferably greater than about 30° C., and in some instances greater than 35° C.

Examples of industrial waste streams that can be used include, but are not limited to, waste streams from coal fired power plants, petroleum refining, tar sand refining, natural gas production, heavy oil upgrading, and/or food processing plants.

### B. A Water Source

The methods disclosed herein can be carried out in any body of water in which the desired microbes can flourish. The body of water can be any size, can be in a closed vessel or an open vessel and/or can include a plurality of open or closed vessels with the use of sunlight and or bioreactors configured for maximum sunlight exposure for algal growth. In one embodiment, the water source is a large open body of water (i.e., a pond). In another embodiment, the water source is a subterranean water associated with a geological formation. The subterranean water can be a large body of water such as an aquifer or the subterranean water can include relatively little standing water and instead be a moist environment capable of supporting microbial growth. In addition, the subterranean water can be a naturally occurring water source or the water can be pumped from a terrestrial source into a subterranean environment. Advantageously, the systems methods disclosed herein can be carried out in a natural environment while still achieving high rates of biomass production.

The body of water can be saline (i.e., a brine water or a nutrient solution rich in biochemical precursors) or fresh water. A brine water can be advantageous to limit the types of lgae and bacteria that can flourish in the body of water. That is, high salt environments kill most species of bacteria, archaea, and algae. In contrast, organisms adapted to such environments (i.e., halophiles) are able to thrive in such environments. The salt concentration can be optimized to produce a microbial culture that maximizes biomass production and/or nutrient uptake. Where a brine body of water is used, the salt concentration can range from about 5 parts-per-thousand (ppt) to 300 ppt, more preferably about 10 ppt to 280 ppt, and most preferably from about 40 ppt to about 250 ppt. In one embodiment, the brine water has a salt concentration that is greater than typical seawater, which has a salt concentration of 35 ppt. Preferably the body of water has a salt concentration greater than about 40 ppt and more preferably greater than about 50 ppt.

Likewise, one will appreciate that the body of water can have a relatively low temperature (e.g., about 35° C.) and a relatively neutral pH (e.g., a pH of about 6-8) or the temperature can be much higher and/or the pH much more extreme. As with brine, high temperature of pH can be advantageous to limit the types of algae and bacteria that can flourish in the body of water. Most microorganisms thrive at a relatively low temperature and in a relatively neutral pH range. In contrast, species of extremophiles are know that thrive at extreme pH (e.g., pH 2 or 10) and/or temperature (e.g., above about 80° C.).

C. Mixing Nutrients with a Body of Water

The industrial waste stream can be mixed with the body of water using any technique that provides high surface area and intimate contact between the water and the industrial waste stream. Examples of devices that can be used to efficiently mix gaseous components with water are known in the art. Devices that can be used to mix the industrial waste stream and the water include, but are not limited to, water-air mixers and micro-bubble infusers. The industrial stream can be injected as a homogeneous mixture or alternatively as a heterogeneous mixture. The industrial stream can be injected as a gas or a liquid. If the industrial stream is injected as a gas, it can be advantageous to inject the gaseous industrial stream so as to create two phase injection stream. Preferably the gaseous industrial stream is injected into the body of water so as to form an emulsion or dispersion of gas and liquids. The dispersion of gaseous industrial stream preferably includes micro-bubbles to ensure high surface area contact. An example of a micro-bubble injector that can be used in the system and methods disclosed herein is described in U.S. Pat. No. 6,763,947, which is hereby incorporated herein by reference. U.S. Pat. No. 6,763,947 describes a flotation separation apparatus for separating and classifying diverse, liquid-suspended solids having a plurality of high volume air bubble infusers. Each infuser includes a circular cavity defined by an interior circumferential wall. A plurality of stationary impinging plates projecting from the interior circumferential wall into the circular cavity and equally spaced circumferentially in series therealong. An injecting stream of water and air impinges upon the impinging plates in series to repeatedly create, divide, and subdivide air bubbles as the injection stream transverses the series of impinging plates.

Other devices that can be used to inject a gaseous or liquid stream into the body of water include, but are not limited to, air sparged hydrocyclones and thin film rotating cylinder systems. An example air sparged hydrocyclone is available from Kemco Systems (Clearwater Fla.). An example thin film rotating treatment system is available from Ionic Water Technologies (Reno, Nev.).

In one embodiment, the waste stream is injected under pressure. The injection pressure can range from about 1 psi to about 100 psi, more preferably about 5 psi to about 50 psi.

The mixture of the waste stream with the body of water provides a nutrient enriched body of water suitable for growing the desired type of microbes. The mixture preferably has the waste stream pollutants (i.e., the salts and particulates that act as the microbial nutrients) sufficiently suspended in the body of water so as to allow the microbes sufficient time to absorb a significant quantity of the nutrients.

In one embodiment, the mixing the industrial waste stream with the body of water increases the salt concentration in the body of water. In this embodiment, a brine is formed, at least in part, from the mixing of the industrial waste stream with the water. In one embodiment, a brine water is formed from a sea water that is mixed with an industrial waste stream to create a brine with a significantly higher salt concentration compared to common seawater.

### D. Microorganisms

The microbes used in the systems and methods described herein are selected to optimize pollution remediation (e.g., the sequestration of CO₂, NOₓ, SOₓ, and heavy metals) and bio-energy production (e.g., biomass and/or biotic methane) in the particular environment created by mixing the industrial waste stream and the body of water. Typically the body of water, the industrial waste stream, the additional nutrients, and the microbes are selected to produce a system that optimizes the production of biomass for a given cost.

The microbes can include algae and/or bacteria and/or archaea. In many cases, a number of species of algae, bacteria, and archaea can be present. The relative ratios of algae to bacteria, bacteria to bacteria, bacteria to archaea, etc. can be controlled to some extent by selecting a ratio of nutrients and the energy source (e.g., sunlight or a hydrocarbon source such as coal) that favors one microbes or a class of microbes over another. In many cases, there exists a symbiotic or mutually beneficial relationship among the microbial population. For example, by optimizing the growing environment (e.g., nutrients, energy source, types of microbes, etc) it is possible to dramatically increase the microbial load in a given environment and, thus, the generation of bio-energy (e.g., biomass or biotic methane).

In a preferred embodiment, the microbes can be brine tolerant, pH tolerant, and/or heat tolerant. In one embodiment, the algae and/or bacteria and/or archaea are halophiles, which are extremophiles that thrive in high salt and metal laden environments. The use of a halophile in combination with a highly saline body of water eliminates a substantial portion of the naturally occurring algae and/or bacteria that could otherwise compete with the desired microbes for the nutrient resources in the nutrient-enriched body of water. Thus, contamination in brine waters is less likely and/or can be more easily controlled.

In one embodiment, the algae and/or bacteria can be selected for specific traits or genetically engineered. The genetic engineering can be used, for example, to up-regulate lipid producing capabilities of the algae or bacteria and/or down regulate other biological mechanisms that reduce the yield of biofuels from the harvested biomass. For example, the microbes can be selected and/or genetically engineered to produce high concentrations of a fuel precursor compounds selected from the group of glycerol, lignins, lipids, and the like, and combinations thereof. Genetically engineered algae and/or bacteria and/or archaea are preferably used, but not required, in combination with a saline body of water to limit microbial competition from natural contamination.

Examples of suitable algae that can be used with the present invention include algae from the genus Dunaliella, such as, but not limited to, Dunaliella salina. A number of bacterial and archael species can be used in the systems and methods described herein. In one example Rhodococcus, Bacillus, Pseudomonas, Clostridia, Burkholderia, Proteobacteria such as Oceanospirillum, Neptunomonas, Alcanivorax, and the like are useful for surfactant production. In another example, Acetobacter sp., Acidiothiobacillus sp., Sulfate Reducing Bacterial sp., Acetobacterium sp., Clostridia sp., Pseudomonas sp., Bacillus sp. and the like are useful for acid production. And in yet another example, Halobacterium is a group of Archaea that contains the genus Halococcus and others that have a high tolerance for elevated levels of salinity. Some species of halobacteria have acidic proteins that resist the denaturing effects of salts. Chromohalobacter is another species. In addition, species such as Methanosarcina sp. Methanococcus sp., Sulfate Reducing Bacterial sp., Acetobacterium, sp., Clostridia sp., Pseudomonas sp., Bacillus sp. and other microbes (Micrococcus, Achromobacter, Flavobacterium, Bacterioides, Serratia, Alcaligenes, and Cellulomonas.) have been found in high salt environments performing degradations of that are believed to be involved in the various complex carbon compound breakdown and in coal bed methane production.

All microbes require nutrient components, C:N:P:S:vitamins: and others, in specific ratios to grow and metabolize various organics. Any imbalance in these ratios can slow or halt microbial growth; thus the breakdown of complex carbons into the simple carbon compounds required for methane formation. Flue gases containing SOx, NOx, CO₂ and other components can provide these nutrients in liquid form or as aerosols. Microbes isolated from high salt environments have demonstrated the conversion of heavy and waste oils, oil sands, and oil shales into methane. Microbes tolerant to higher salt and ionic strength are needed to grow in the solutions that utilize high concentrations of flue gases as nutrients and in coal bed methane environments. FIG. 3B illustrates various processes and steps in the degradation of complex hydrocarbons (e.g., coal) for the production of methane.

Considerable enhancement of microbial growth, metabolism of organic geopolymers, and methane production can be obtained by providing and maintaining the proper microbial population, environmental, and nutrient component balance. For example, with the addition (e.g., staged and/or cyclical addition) of appropriate microbes, nutrients, and environmental adjustment components at various points, significant increases in methane production should be possible.

As illustrated in FIG. 3B, methanogenesis from sources such as coal often involves a consortium of microorganisms to convert the geopolymers in fossil fuels to simple carbon compounds from which methane can be produced. As a whole, microbial methane production typically involves hydrolytic fermentative microbes that convert geopolymers in coal, shale and/or petroleum into colloidal polymers. Hydrolytic fermentative microbes then convert the colloidal polymers to fatty acids, sugars, amino acids, ammonia, hydrogen sulfide, carbon dioxide, acetate, acid, and combinations of these. Syntrophic acetogenic microbes convert these components to acetate, hydrogen, and carbon dioxide, which are then consumed by methanogenic microbes to produce methane and carbon dioxide.

In this process, methane is produced by carbonate reduction and fermentation. Fermentation and carbonate reduction proceed according to the following two reactions, respectively:

CO₂+4H₂→4CH₄+2H₂O

CH₃COO⁻+H₂O→CH₄+HCO₃⁻

Significant increases in methane production from geological sources (e.g., coal beds, and/or depleted oil reserves) are possible with the addition of selected microbes and/or nutrients and adjustment of environmental conditions. For example, numbers of acidophilic, hydrolytic, and fermentative bacteria/archaea involved in the breakdown of carbon polymers in coal, oil shale, tar sands, and heavy oils to various humic acids and other colloidal polymers and hydrolytic and fermentative bacteria/archaea involved in the breakdown of various humic acids and other colloidal polymers to fatty acids, sugars, amino acids (i.e., the first and second steps in the scheme shown in FIG. 3B) are typically limiting in most subterranean environments where methane production from coal, oil shale, heavy oil, and the like is desired. Likewise, addition of components of flue gases (e.g., SOₓ, NOₓ, CO₂, etc.) at various points can produce solutions or environments in which significant increases in methane production may be obtained.

In one embodiment, the microbes injected into the geological formation are extremeophiles. The microorganisms may be provided by culturing naturally occurring bacteria and archaea under appropriate conditions to optimize the overall environment in which biotic methane will be produced. The conditions can facilitate self selection of microbial populations that are most suited for producing methane in the harsh environment of the geological formation. Different combinations of bacteria and archaea populations can be used in the selection process. Microoganisms are the earliest forms of life on earth and occupy almost every conceivable ecological niche, even the harshest, most extreme, and toxic environments. About 1 billion microbes live in a single teaspoon of moist garden soil. In a preferred embodiment, the microbial population is enriched with extremophiles including halophiles and thermophiles, which are microbes that thrive in higher salt, ionic strength, and higher-temperature environments, respectively. Using a variety of naturally occurring bacteria and archaea in combination with selection pressures allows the most robust population of microbes to be obtained. However, the systems and methods disclosed herein may also be carried out using engineered microbes alone or in combination with naturally occurring microbes. Where an engineered microbe is used, the engineered microbes may be produced from one or more of the foregoing naturally occurring microbes that have been selected to provide robust methane production in the geological formation.

### E. Recovering Biomass and Production of Biofuels

The rapid growth of the algae and/or bacteria and/or archaea produces a biomass that is useful as an energy source. At least a portion of the microbes are recovered by separating the microbes from the body of water and processing the microbes to obtain a useful product. In one embodiment, the recovered microbes are processed into a biofuel such as biodiesel or a light hydrocarbon such as methane. The algae and/or bacteria and/or archaea can also be selected and grown to produce methane or a similar hydrocarbon that is useful directly as a biofuel.

Flue gas nutrients can be used to produce microbes that are a source of biomass, enzymes, microorganisms, and other carbon materials for a broad spectrum of nutrients. These materials can be utilized in surface ponds or reactors (ex situ), through introduction into solutions as aerosols, to stimulate a variety of microorganisms and algae for the produce a variety of biofuels including methane. Because in many systems, the bacterial and algal populations are syntropic, the amount of bacterial biomass can be equal to or significantly larger than other forms of biomass produced, it is important to optimize both bacterial and algal biomass production for energy production.

Algae and cyanobacteria and other bacteria (bacteria, algae, and archaea) have been studied extensively in the past, mainly as a source of protein (animal feed, vitamins and other food supplements) and fuel and most large scale operations utilize open outdoor ponds or raceways to produce large quantities of these organisms. One of the more interesting properties of these organisms is their ability to convert carbon dioxide and grow under conditions usually not conducive to cell growth, such as in high salinity and ionic strength brines. Thus, it is possible to use these properties in the removal of CO₂, SOₓ, and NOₓ from flue gases.

While much work has been reported on the growth characteristics of these microbes individually, the inventor is not aware of work focusing on enhancing the growth characteristics of these microbes as a community (i.e., symbiotic growth) to increase the total mass of biomass or optimizing this biomass for biofuel production. Because nutrient solutions based systems sequestering flue gases ultimately end up as brine solutions (i.e., nutrient rich solutions having a high ionic strength), it is important to examine algae and bacteria naturally found at high levels in these higher salt systems. Laboratory tests performed by the inventor in this case have demonstrated that it is possible to significantly increase biomass yields with appropriate adjustments to the consortia of organisms, nutrients, and environmental adjustments.

Flue gasses can also be utilized in liquid or aerosol form to stimulate a broad community of microorganisms for production of microbes and enzymes (e.g., from lysed cells) with different metabolic capabilities for transformation of a broader spectrum of complex organic substances that will provide the most effective in situ transformation kinetics for end product methane formation. Aerosol introduction of all materials and microbes involved is important because of the potential for greater penetration into in situ formations. These microbes can work in synergy with microbial enzyme preparations and chemicals produced by the use of flue gases as nutrients like sulfuric, hydrochloric, and nitric acids. Brines and nutrient solutions produced using flue gases and other additives like sugars produce the protein and sugar rich environments that stabilize both microbes and enzyme preparations during aerosolization and injection into in situ formations.

In one embodiment, a biofuel is produced by collecting a portion of the biomass in the body of water and concentrating the biomass. Concentration can be carried out using a centrifuge or other technique that separates water from the biomass. In one embodiment the biomass is processed to extract lipids. Typically the extracted lipids are in the form of a fatty acid. The fatty acids can be converted to biodiesel and glycerol using transesterification. Those skilled in the art are familiar with these and other processes for producing hydrocarbons from the fatty acids found in a biomass. The biodiesel can also be upgraded to other hydrocarbons such as, but not limited to, jet fuel, gasoline, lubricants, DMF, and the like using techniques known in the petroleum refining industry. An example of process that can be used to convert a lipidic biomass to a hydrocarbon fuel is disclosed in U.S. patent application publication number 2009/0069610, which is hereby incorporated herein by reference.

For purposes of this invention, the term “Industrial Waste Stream” includes, but is not limited to, fluid streams that have nutrients that are beneficial to microorganisms but that include compounds and/or concentrations of compounds that require treatment before being released into a natural environment.

### F. Methods

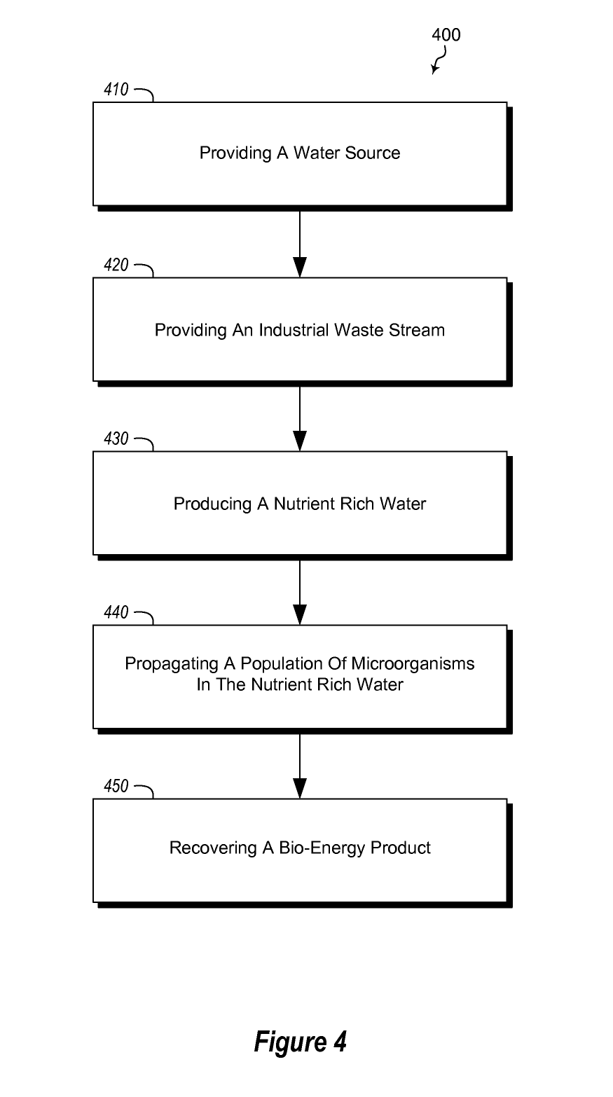

Referring now to FIG. 4, a flow diagram of a method 400 for bio-energy recovery and/or production using an industrial waste stream and a microbial population is illustrated. The method 400 includes (1) providing an industrial waste stream 420 that includes nutrient compounds capable providing a nutrient rich environment when mixed with water (e.g., a water source provided in act 410), (2) mixing the industrial waste stream with a water source 430 to produce a nutrient rich water, (3) propagating a population of microorganisms 440 including at least one of algae, archaea, bacteria, methanogenic microorganisms, pH tolerant microbes, heat tolerant microbes, and/or brine tolerant microbes in the nutrient rich water, and (4) recovering a bio-energy product 450 produced by the population of microorganisms.

Suitable examples of water sources include, but are not limited to, natural and artificial lakes, ponds, and the like and generally moist environments that can support microbial growth. In one aspect, the water source can be a surface body of water such as a pond, a lake, or a waste lagoon. In another aspect, the water source can be a subterranean body of water such as a body of water associated with a geological formation that includes a hydrocarbon material.

In one embodiment, the subterranean water is injected into a geological formation that includes a hydrocarbon material. In one example, the method further includes injecting at least a portion of the nutrient rich water into a geological formation that includes a hydrocarbon material, allowing the population of microorganisms to propagate using the hydrocarbon material and nutrient compounds from the industrial waste stream, and recovering the bio-energy product from the geological formation. In one embodiment, the injecting can include at least one of injecting a liquid water into the geological formation or injecting an aerosol into the geological formation.

In one embodiment, at least some portion of the microbes in the microbial population propagated in action 420 can be indigenous to the water source and/or the geological formation. For example, the microbial population can include at least one methanogenic microorganism capable of producing a biotic methane in the geological formation. In another example, the microbial population can include at least one microbe that produces at least one precursor (e.g., acetate) that can be utilized by the methanogenic microorganism for methane production.

In one embodiment, the microbial population propagated in action 420 includes at least about 10% by weight of bacteria. In one embodiment, the bacteria is a halobacterium.

In one embodiment, the bio-energy product includes a biomass. Accordingly, the method may further include processing the biomass to produce a hydrocarbon fuel such as, but not limited to, biodiesel, jet fuel, 2,5-Dimethylfuran (DMF), alcohol, and the like.

In another embodiment, the biomass can be processed to recover heavy metals such as, but not limited to, mercury, lead, and cadmium that are component of typical flue gases.

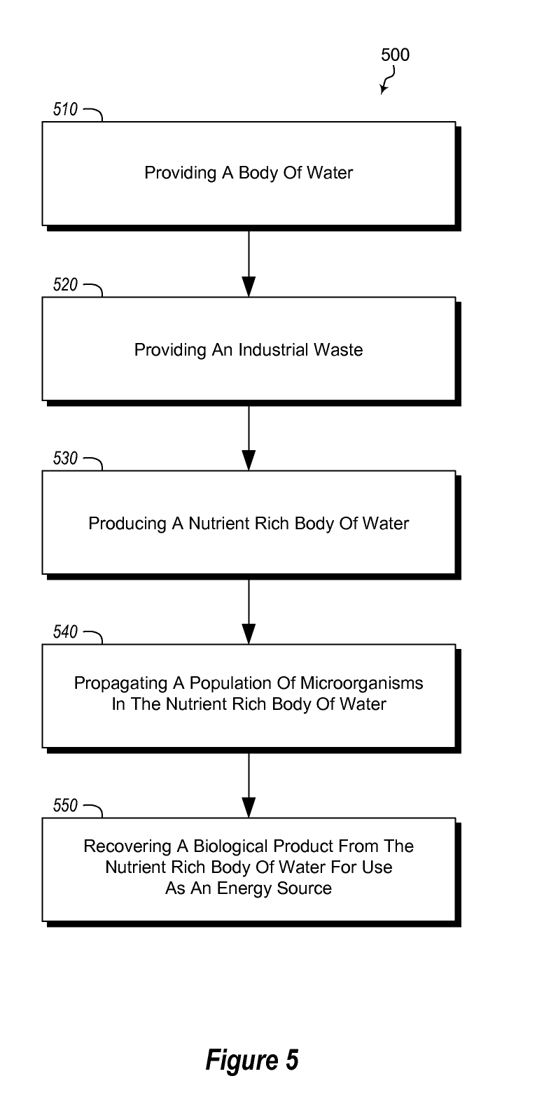

Referring now to FIG. 5, a flow diagram of a method for bio-energy recovery and/or production using an industrial waste stream, sunlight, and a microbial population is illustrated. The method includes (1) providing a body of water 510, (2) providing an industrial waste stream 520 including nutrient compounds capable of providing a nutrient rich environment when mixed with water, (3) mixing the industrial waste stream with the body of water 530 to produce a nutrient rich body of water, (4) propagating a bacterial and/or algal population in the nutrient rich body of water in the presence of sunlight 540, wherein the bacterial and/or algal population includes algae, archaea, bacteria, methanogenic microorganisms, pH tolerant microbes, heat tolerant microbes, and/or brine tolerant microbes, and combinations thereof, and (5) recovering a biological product 550 from the nutrient rich body of water for use as an energy source.

In the present method, sunlight is utilized as an energy source to supplement growth of the microbial culture. As in the previously described method, the microbial population can be configured for production of a biofuel such as methane, production of a biomass that can be processed to yield a biofuel, and/or for heavy metal recovery. In one example, the microbial population can include Dunaliella or Cyanophyta for sequestration of CO₂, SOₓ and/or NOₓ. In another example, the bacterial and/or algal population is configured to produce high concentrations of a fuel precursor compounds selected from the group of glycerol, lignins, lipids and combinations thereof.

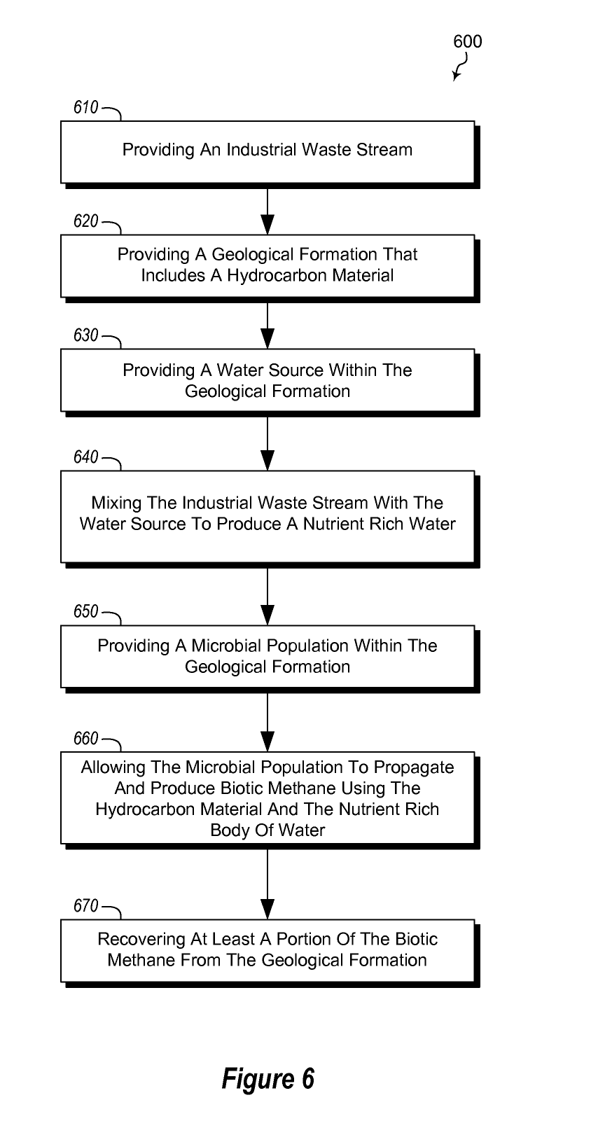

Referring now to FIG. 6, a flow diagram of a method for producing biotic methane in a geological formation using an industrial waste stream and a microbial population is illustrated. In one embodiment, the method includes (1) providing an industrial waste stream 610 including nutrient compounds capable of providing a nutrient rich environment when mixed with water, (2) providing a geological formation 620 that includes a hydrocarbon material, (3) providing a water source 630 within the geological formation, (4) mixing the industrial waste stream with the water source 640 to produce a nutrient rich water, (5) providing a microbial population and/or one or more microbial components within the geological formation 650, (6) allowing the microbial population to propagate and produce biotic methane 660 using the hydrocarbon material and the nutrient rich water, and (7) recovering at least a portion of the biotic methane 670 from the geological formation.

In one aspect, the microbial population provided within geological formation includes at least one of archaea, bacteria, methanogenic microorganisms, pH tolerant microbes, heat tolerant microbes, and/or brine tolerant microbes, and combinations thereof. According to the present invention, the microbial population provided within the geological formation can be a microbial population that is indigenous to the geological formation or indigenous to a similar geological formulation, or the microbial population can be augmented microbial consortia that is propagated in the nutrient rich body of water and subsequently injected in whole or in part into the geological formation. The microbial population can, for example, be injected into the geological formation in a liquid water or as an aerosol. In one embodiment, the aerosol may include at least one of an aqueous portion, a nutrient potion derived from the industrial waste stream, and/or a microbial portion derived from the microbial population. Microbes and solutions containing microbes can be aerosolized using a number of techniques known in the art. For example, the microbial population can be concentrated by, for example, centrifugation and subsequently mixed with a protectant such as a sugar or a protein that can protect the microbes from dehydration while in the aerosol state.

Referring again to FIG. 3B, one will appreciate that different stages of the process of bioconverting carbon polymers in coal, oil shale, tar sands, heavy oils, and the like into methane involves a number of stages that depend on different organisms that may require different nutrients at different stages. As a whole, microbial methane production typically involves acidophilic and hydrolytic fermentative microbes that convert geopolymers in coal, shale and/or petroleum into colloidal polymers. Hydrolytic fermentative microbes then convert the colloidal polymers to fatty acids, sugars, amino acids, ammonia, hydrogen sulfide, carbon dioxide, acetate, acid, and combinations of these. Syntrophic acetogenic microbes convert these components to acetate, hydrogen, and carbon dioxide, which are then consumed by methanogenic microbes to produce methane and a number of other metabolites.

As such, in one embodiment, the method can further include (i) providing a first microbial population and a first nutrient and/or a first microbial component within the geological formation, and (ii) providing at least a second microbial population and at least a second nutrient and/or a second microbial component within the geological formation. The first nutrient and the at least second nutrient are derived from the industrial waste stream, wherein the first nutrient and the at least second nutrient are the same or different.

In one aspect, the first microbial population and/or the one or more microbial components are configured to degrade the hydrocarbon material in the geological formation to produce humic acids and/or colloidal polymers and the at least second microbial population and/or the second microbial component are configured to degrade the humic acids and/or colloidal polymers to produce one or more of fatty acids, sugars, amino acids, ammonia, H₂S, hydrogen, acetate, and methane. In another aspect, the second microbial population and/or the second microbial component can include microbes and/or components that convert the humic acids and colloidal polymers produced by the first microbial population and/or the first cellular component to fatty acids, sugars, and amino acids, and that convert the fatty acids, sugars, and amino acids to ammonia, H₂S, CO₂, hydrogen, acetate, and other compounds that can be utilized by methanogens for the production of methane. Microbes, cellular components, and nutrients needed for bioconversion of carbon polymers to methane can be delivered all at once or in a staged and/or cyclic fashion in order enhance methane production.

In one embodiment, the one or more microbial components include enzymes and surfactants adapted to facilitate biotic methane production and/or microbial growth in the geological formation. The one or more microbial components (e.g., enzymes and surfactants) are typically derived from the microbial population.

In one embodiment, the water source provided within the geological formation can be a surface water that is injected into the geological formation, or the body of water can be a subsurface body of water that is associated with the geological formation. For example, a nutrient rich water can be prepared on the earth's surface and injected into the geological formation. In another embodiment, the nutrient rich water can be prepared or adjusted in situ in the geological formation by injecting the industrial waste stream (e.g., a flue gas) into the geological formation where it will mix with water naturally present in the geological formation or with water supplied in the geological formation.

In one embodiment, the geological formation is a coal bed, an oil shale bed, depleted oil field, a tar sand deposit, and the like. In one embodiment, the hydrocarbon material has been fractured. In one embodiment, the method further includes injecting a fracturing fluid into the geological formation to crack the hydrocarbon material and increase the surface area thereof. In one embodiment, the fracturing fluid includes carbon dioxide and or other flue gases and nutrients. In another embodiment, the fracturing fluid includes a hydrocarbon. An example of a hydrocarbon that may be injected into the geological formation includes, but is not limited to, acetate, lactate, methanol, and/or ethanol.

The present invention may be embodied in other specific forms without departing from its spirit or essential characteristics. The described embodiments are to be considered in all respects only as illustrative and not restrictive. The scope of the invention is, therefore, indicated by the appended claims rather than by the foregoing description. All changes which come within the meaning and range of equivalency of the claims are to be embraced within their scope.

What is claimed is:

1. A method, comprising:
providing an industrial waste stream including nutrient compounds capable providing a nutrient rich environment when mixed with water;
mixing the industrial waste stream with a water source to produce a nutrient rich water;
propagating a population of microorganisms including at least one of algae, archaea, bacteria, methanogenic microorganisms, pH tolerant microbes, heat tolerant microbes, and/or brine tolerant microbes in the nutrient rich water; and
recovering a bio-energy product produced by the population of microorganisms.

2. The method of claim 1, wherein the water source is a surface water.

3. The method of claim 2, wherein the surface water is at least one of a pond, a lake, or a waste lagoon.

4. The method of claim 1, wherein the water source is a subterranean water.

5. The method of claim 4, wherein the subterranean water is associated with a geological formation that includes a hydrocarbon material.

6. The method of claim 4, wherein the subterranean water is injected into a geological formation that includes a hydrocarbon material.

7. The method of claim 1, further comprising:
injecting at least a portion of the nutrient rich water into a geological formation that includes a hydrocarbon material;
allowing the population of microorganisms to propagate using the hydrocarbon material and nutrient compounds from the industrial waste stream; and
recovering the bio-energy product from the geological formation.

8. The method of claim 7, wherein the injecting includes at least one of injecting a liquid water into the geological formation or injecting an aerosol into the geological formation.

9. The method of claim 8, wherein at least some microbes in the microbial population are indigenous to the geological formation.

10. The method of claim 8, wherein the microbial population includes at least one methanogenic microorganism capable of producing a biotic methane in the geological formation.

11. The method of claim 10, further comprising recovering at least a portion of the biotic methane from the geological formation.

12. The method of claim 1, wherein the industrial waste stream is a gaseous waste stream.

13. The method of claim 12, wherein the gaseous industrial waste stream is a flue gas from an industrial power plant.

14. The method of claim 13, wherein the industrial power plant is a coal fired power plant.

15. The method of claim 1, wherein the industrial waste stream is a liquid waste stream.

16. The method of claim 1, wherein the industrial waste stream is from a food processing plant.

17. The method of claim 1, wherein the salt concentration in the nutrient rich water is about 35 parts-per-thousand (ppt).

18. The method of claim 1, wherein the salt concentration in the nutrient rich water is greater than about 40 ppt.

19. The method of claim 1, wherein the salt concentration in the nutrient rich water is greater than about 50 ppt.

20. The method of claim 1, wherein the salt concentration in the nutrient rich water is in a range from about 10 ppt to about 300 ppt.

21. The method of claim 1, wherein the nutrient rich water has a carbon content in a range from about 100-120 mg/l.

22. The method of claim 1, wherein the nutrient rich water has a nitrogen content in a range from about 10-20 mg/l.

23. The method of claim 1, wherein the nutrient rich water has a phosphorus content in a range from about 1-3 mg/l.

24. The method of claim 1, wherein the nutrient rich water has a sulfur content in a range from about 1-2 mg/l.

25. The method of claim 1, wherein the population of microorganisms includes at least 10% by weight algae.

26. The method of claim 25, wherein the algae belong to the genus Dunaliella.

27. The method of claim 1, wherein the population of microorganisms includes at least about 10% by weight of bacteria.

28. The method of claim 27, wherein the bacteria is a halobacterium.

29. The method of claim 1, wherein the bio-energy product comprises biomass.

30. The method of claim 29, further comprising processing the biomass to produce a hydrocarbon fuel.

31. The method of claim 29, further comprising processing the biomass to recover heavy metals.

32. The method of claim 1, wherein the industrial waste stream has a temperature equal to or greater than that of the water.

33. The method of claim 1, wherein the industrial waste stream is mixed with the water using an infuser system.

34. A method, comprising:
providing a body of water;
providing an industrial waste stream including nutrient compounds capable of providing a nutrient rich environment when mixed with water;
mixing the industrial waste stream with the body of water to produce a nutrient rich body of water;
propagating a bacterial and/or algal population in the nutrient rich body of water in the presence of sunlight, wherein the bacterial and/or algal population comprises at least one of algae, archaea, bacteria, methanogenic microorganisms, pH tolerant microbes, heat tolerant microbes, and/or brine tolerant microbes, and combinations thereof; and
recovering a biological product from the nutrient rich body of water for use as an energy source.

35. The method of claim 34, wherein the industrial waste stream is a gaseous fluid stream.

36. The method of claim 35, wherein the gaseous fluid stream is a flue gas from an industrial power plant.

37. The method of claim 36, wherein the industrial power plant is a coal fired power plant.

38. The method of claim 34, wherein the industrial waste stream is a liquid waste stream.

39. The method of claim 34, wherein the industrial waste stream is from a food processing plant.

40. The method of claim 34, wherein the industrial waste stream is a liquid.

41. The method of claim 34, wherein the nutrient rich body of water has a salt concentration greater than about 40 ppt.

42. The method of claim 34, wherein the nutrient rich body of water has a salt concentration greater than about 50 ppt.

43. The method of claim 34, wherein the nutrient rich body of water has a salt concentration in a range from about 10 ppt to about 300 ppt.

44. The method of claim 34, wherein the nutrient rich body of water has a salt concentration in a range from about 100-120 mg/l.

45. The method of claim 34, wherein the nutrient rich body of water has a nitrogen content in a range from about 10-20 mg/l.

46. The method of claim 34, wherein the nutrient rich body of water has a phosphorus content in a range from about 1-3 mg/l.

47. The method of claim 34, wherein the nutrient rich body of water has a sulfur content in a range from about 1-2 mg/l.

48. The method of claim 34, wherein at least 10% by weight of microorganisms in the nutrient rich body of water are algae.

49. The method of claim 48, wherein the algae belong to the genus Dunaliella.

50. The method of claim 34, further comprising:
recovering at least a portion of the bacterial and/or algal population from the nutrient rich body of water; and
processing the recovered portion of the bacterial and/or algal population to recover heavy metals.

51. The method of claim 34, wherein the industrial waste stream has a temperature equal to or greater than the body of water.

52. The method of claim 34, wherein the body of water is a pond, a lake, or a waste lagoon.

53. The method of claim 34, wherein sunlight is used to supplement microbial and or algal growth.

54. The method of claim 34, wherein bacterial and/or algal population includes Cyanophyta for sequestration of CO₂, SOₓ and/or NOₓ.

55. The method of claim 34, wherein the bacterial and/or algal population is configured to produce high concentrations of a fuel precursor compounds selected from the group of glycerol, lignins, lipids and combinations thereof.

56. The method of claim 34, wherein the biological product includes a biomass used to produce biofuel.

57. A method for producing biotic methane, comprising:
providing an industrial waste stream including nutrient compounds capable of providing a nutrient rich environment when mixed with water;
providing a geological formation that includes a hydrocarbon material;
providing a water source within the geological formation;
mixing the industrial waste stream with the water source to produce a nutrient rich water;
providing a microbial population and/or one or more microbial components within the geological formation;
allowing the microbial population to propagate and produce biotic methane using the hydrocarbon material and the nutrient rich water; and
recovering at least a portion of the biotic methane from the geological formation.

58. The method of claim 57, wherein the microbial population includes archaea, bacteria, methanogenic microorganisms, pH tolerant microbes, heat tolerant microbes, and/or brine tolerant microbes, and combinations thereof.

59. The method of claim 57, wherein at least some microbes in the microbial population are indigenous to the geological formation.

60. The method of claim 57, wherein the one or more microbial components include enzymes and surfactants adapted to facilitate biotic methane production and/or microbial growth in the geological formation.

61. The method of claim 60, wherein the one or more microbial components are derived from the microbial population.

62. The method of claim 57, further comprising:
providing a first microbial population and a first nutrient and/or a first microbial component within the geological formation; and
providing at least a second microbial population and at least a second nutrient and/or a second microbial component within the geological formation.

63. The method of claim 62, wherein the first nutrient and the at least second nutrient are derived from the industrial waste stream.

64. The method of claim 62, wherein the first nutrient and the at least second nutrient are the same or different.

65. The method of claim 62, wherein the first microbial population and/or the one or more microbial components are configured to degrade the hydrocarbon material in the geological formation to produce humic acids and/or colloidal polymers.

66. The method of claim 65, wherein the at least second microbial population and/or the second microbial component are configured to degrade the humic acids and/or colloidal polymers to produce one or more of fatty acids, sugars, amino acids, ammonia, H₂S, hydrogen, acetate, and methane.

67. The method of claim 57, wherein the geological formation is a coal bed, an oil shale bed, depleted oil field, or a tar sand deposit.

68. The method of claim 57, wherein the hydrocarbon material has been fractured.

69. The method of claim 57 further comprising injecting a fracturing fluid into the geological formation to crack the hydrocarbon material and increase the surface area thereof.

70. The method of claim 57, wherein the fracturing fluid includes carbon dioxide.

71. The method of claim 57, wherein the fracturing fluid includes a hydrocarbon.

72. The method of claim 57, wherein the providing the microbial population within the geological formation includes injecting at least a portion of the microbial population into the geological formation.

73. The method of claim 57, wherein the microbial population includes one or more extremophiles.

74. The method of claim 73, wherein the one or more of the extremophiles includes one or more halophiles.

75. The method of claim 57, wherein the industrial waste stream provides at least one of carbonates, sulfates, phosphates, transition metals, and combinations of these.

76. The method of claim 57, wherein the industrial waste stream is a gaseous waste stream.

77. The method of claim 76, wherein the gaseous industrial waste stream is a flue gas from an industrial power plant.

78. The method of claim 77, wherein the industrial power plant is a coal fired power plant.

79. The method of claim 57, wherein the waste stream is a liquid waste stream.

80. The method of claim 57, wherein the nutrient rich water is a salt or brine water.

81. The method of claim 80, wherein the salt concentration in the brine water is greater than about 40 ppt.

82. The method of claim 80, wherein the salt concentration in the brine water is greater than about 50 ppt.

83. The method of claim 80, wherein the salt concentration in the brine water is in a range from about 10 ppt to about 300 ppt.

84. The method of claim 57, wherein the nutrient rich water has a carbon content in a range from about 100-120 mg/l.

85. The method of claim 57, wherein the nutrient rich water has a nitrogen content in a range from about 10-20 mg/l.

86. The method of claim 57, wherein the nutrient rich water has a phosphorus content in a range from about 1-3 mg/l.

87. The method of claim 57, wherein the nutrient rich water has a sulfur content in a range from about 1-2 mg/l.

88. The method of claim 57, wherein the industrial waste stream has a temperature equal to or greater than a water in the geological formation.

89. The method of claim 57, further comprising providing a nutrient rich water within the geological formation.

90. The method of claim 89, wherein the providing includes at least one of injecting the nutrient rich water into the geological formation or forming the nutrient rich water within the geological formation.

91. The method of claim 90, wherein the injecting includes at least one of injecting a liquid water into the geological formation or injecting an aerosol into the geological formation.

92. The method of claim 91, wherein the aerosol includes at least one of an aqueous portion, a nutrient potion derived from the industrial waste stream, and/or a microbial portion derived from the microbial population.

93. A system for bio-energy recovery and/or production, comprising:
a water source;
an industrial waste stream including nutrient compounds capable of producing a nutrient rich water when admixed with water;
a population of microorganisms configured to propagate in the nutrient rich water, wherein the population of microbes includes at least one of algae, archaea, bacteria, methanogenic microorganisms, pH tolerant microbes, heat tolerant microbes, and/or brine tolerant microbes.

94. The system of claim 93, wherein the water source comprises fresh water, brackish water, or salt water.

95. The system of claim 93, wherein the water source is a surface body of water.

96. The system of claim 90, wherein the surface body of water is at least one of a pond, a lake, or a waste lagoon.

97. The system of claim 93, wherein the water source is a subterranean body of water.

98. The system of claim 97, wherein the subterranean body of water is associated with a geologic formation that includes a hydrocarbon material.

99. The system of claim 97, wherein the subterranean body of water is injected into a subterranean geological formation that includes a hydrocarbon material.

100. The system of claim 99, wherein the subterranean geological formation includes an indigenous population of microorganisms.

101. The system of claim 100, wherein the indigenous population of microorganisms includes at least one methanogenic microorganism capable of producing a biotic methane in the subterranean geological formation using the industrial waste stream and the hydrocarbon material.

102. The system of claim 100, wherein the indigenous population of microorganisms includes at least one microorganism that is adapted to produce a methane precursor material for the at least one methanogenic microorganism.

103. The system of claim 99, the nutrient rich water further including the population of microorganisms, wherein the population of microorganisms is adapted for propagation in the subterranean geological formation.

104. The system of claim 93, wherein the industrial waste stream is a gaseous waste stream.

105. The system of claim 104, wherein the gaseous industrial waste stream is a flue gas from an industrial power plant.

106. The system of claim 105, wherein the industrial power plant is a coal fired power plant.

107. The system of claim 93, wherein the industrial waste stream is a liquid waste stream.

108. The system of claim 93, wherein the industrial waste stream is from a food processing plant.

109. The system of claim 93, wherein the salt concentration in the nutrient rich body of water is about 35 parts-per-thousand (ppt).

110. The system of claim 93, wherein the salt concentration in the nutrient rich body of water is greater than about 40 ppt.

111. The system of claim 93, wherein the salt concentration in the nutrient rich body of water is greater than about 50 ppt.

112. The system of claim 93, wherein the salt concentration in the nutrient rich body of water is in a range from about 10 ppt to about 300 ppt.

113. The system of claim 93, wherein the nutrient rich body of water has a carbon content in a range from about 100-120 mg/l.

114. The system of claim 93, wherein the nutrient rich body of water has a nitrogen content in a range from about 10-20 mg/l.

115. The system of claim 93, wherein the nutrient rich body of water has a phosphorus content in a range from about 1-3 mg/l.

116. The system of claim 93, wherein the nutrient rich body of water has a sulfur content in a range from about 1-2 mg/l.

117. The system of claim 93, wherein the population of microorganisms includes at least 10% by weight algae.

118. The system of claim 117, wherein the algae belong to the genus Dunaliella.

119. The system of claim 93, wherein the population of microorganisms includes at least about 10% by weight of bacteria.

120. The system of claim 119, wherein the bacteria is a halobacterium.

121. The system of claim 93, wherein the bio-energy product comprises biomass.

122. The system of claim 121, wherein the biomass is configured to be converted into a hydrocarbon fuel.

123. The system of claim 121, wherein the biomass is configured to be processed to recover heavy metals.

124. The system of claim 93, wherein the industrial waste stream has a temperature equal to or greater than that of the body of water.
